(19) United States
(12) Patent Application Publication
(10) Pub. No.: US 2011/0190523 A1
(43) Pub. Date: Aug. 4, 2011

(54) HYDROGENATION OF ESTERS OR CARBONYL GROUPS WITH PHOSPHINO-OXIDE BASED RUTHENIUM COMPLEXES

(76) Inventors: Lionel Saudan, Geneva (CH); Christophe Saudan, Geneva (CH); Michel Alfred Jose Saudan, Geneva (CH); Sylvia Joyeuse Adélaïde Ada Saudan, Geneva (CH)

(21) Appl. No.: 13/063,385

(22) PCT Filed: Oct. 1, 2009
(86) PCT No.: PCT/IB09/54297

§ 371 Date: Mar. 10, 2011

(30) Foreign Application Priority Data
Oct. 2, 2008 (EP) 08165746.2

(51) Int. Cl. C07C 29/14; C07F 15/00

(52) U.S. Cl. 556/18; 568/814

(57) ABSTRACT

The present invention relates to the field of catalytic hydrogenation and, more particularly, to the use of specific ruthenium catalysts or pre-catalysts in hydrogenation processes for the reduction of ketones, aldehydes, esters or lactones into their corresponding alcohols or diols respectively. The preferred catalysts are ruthenium complexes comprising a ligand of the type (N—N) type and a ligand of the type (P—PO).

### TECHNICAL FIELD

The present invention relates to the field of catalytic hydrogenation and, more particularly, to the use of specific ruthenium catalysts, or pre-catalysts, in hydrogenation processes for the reduction of ketones, aldehydes and esters or lactones into the corresponding alcohol or diol respectively.

### PRIOR ART

The reduction of the C═O bond in a ketone, aldehyde or ester functional group to the corresponding alcohol is one of the fundamental reactions in organic chemistry, and is used in a large number of chemical processes. In general, two main types of processes are known to achieve such a transformation. Such types of processes are the following:
a) hydride processes, in which a silyl or metal hydride salt, such as LiAlH₄, or PMHS (polymethylhydrosiloxane) is used;
b) hydrogenation processes, in which molecular hydrogen is used.

From a practical point of view, hydrogenation processes are more attractive as they can be run using small amounts of catalyst (typically 10 to 1000 ppm relative to the substrate) and in the presence of small quantities or even in the absence of solvent. Furthermore, hydrogenation processes do not require the use of highly reactive and expensive hydrides, and do not produce important amounts of aqueous waste.

One of the mandatory and characterizing elements of hydrogenation processes is the catalyst or the catalytic system which is used to activate the molecular hydrogen in view of the reduction. The development of useful catalysts or catalytic systems for the hydrogenation of a ketone, aldehyde or ester functional group represents an important, difficult and unpredictable task in chemistry.

The widely used and described catalysts or catalytic systems known to perform such reductions are all based on ruthenium complexes containing a P₂N₂ coordination sphere; in particular of the (PP)(NN) type (see EP 0901997 and EP 1813621 for ketone and aldehydes, or more recently WO08/065588 for esters), or (PN)(PN) type (see WO02/022526 or WO02/40155 for ketone or aldehydes and WO2006/106483 or WO2006/106484 for esters).

From the examples cited herein above, one can notice that the catalysts reported for the hydrogenation of a substrate always have a similar coordination sphere (P₂N₂) and there is no example where it used a different coordination sphere (e.g PON₂, as in the present case) providing a larger choice of electronic effect on the metal centre, allowing thus a greater flexibility in the tuning of the activity of the catalyst.

Furthermore, from the examples cited herein above, one can notice that the catalysts reported used a strong base in the catalytic systems, which hampered such catalytic system to be used with base sensitive ketones. Only two catalytic systems have been reported to avoid such a problem (see R. Noyori & al., in J. Am. Chem. Soc. 2002, 124, 6508-6509; J. Am. Chem. Soc. 2006, 128, 8724-8725 or Nagoya Industrial Science Research Institute U.S. Pat. No. 6,720,439 and US 2007/0225528), moreover the first one is based on the synthesis of a non-air stable ruthenium complex and the second one showed a low reactivity.

Ruthenium complexes having a similar structure to the one of the present invention are reported by Cyr et al. (Organometallics, 2002, 4672), However this document does not mention or suggest any particular catalytic activity for the hydrogenation.

In view of the above, there is a need for hydrogenation processes using catalysts or pre-catalysts allowing a greater diversity in the ligand structure allowing additional tuning of the steric and electronic around the metal and moreover which can operate under base free conditions by using easily available ruthenium precursors.

### DESCRIPTION OF THE INVENTION

In order to overcome the problems aforementioned, the present invention relates to processes for the reduction by hydrogenation, using molecular H₂, of a C₃-C₇₀ substrate containing one, two or three ketones, aldehydes and/or ester/lactone functional groups into the corresponding alcohol or diol, characterized in that said process is carried out in the presence of
at least one catalyst or pre-catalyst in the form of a ruthenium complex comprising:
   a C₂₋₄₀ diamino bidentate ligand (N—N) wherein at least one of said amino groups is a secondary or primary amine (i.e. a NH or NH₂) and the nitrogen atom of said amine is bound to hydrogen atoms or sp³ carbon atoms; and
   a C₅₋₅₀ phosphine-(phosphine oxide) bidentate ligand (P—PO ligand); and
optionally of a base.

As well understood by a person skilled in the art, by “diamino bidentate” it is understood that said ligand coordinates the Ru metal with two nitrogen atoms. Similarly, by “phosphine-(phosphine oxide) bidentate” it is understood that said ligand coordinates the Ru metal with one phosphorous atom and one oxygen atom (of the PO group).

As well understood by a person skilled in the art, by “sp³ carbon atoms” it is understood that said carbon atom is not part of a carbon-carbon double or triple bond or of an aromatic group.

According to a particular embodiment of the invention, the substrate can be a C₃₋₃₀ compound, in particular of formula of formula (I)

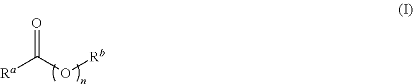

wherein n represents 0 or 1;
R^{a} represents a hydrogen atom or a R^{b} group;
R^{b} represents a C₁-C₃₀ hydrocarbon group, optionally substituted and optionally comprising one, two, three or four heteroatoms selected from the group consisting of oxygen, nitrogen or halogens; or
R^{a} and R^{b}, taken together, represent a C₃-C₂₀, preferably C₄-C₂₀, saturated or unsaturated hydrocarbon group, optionally substituted and optionally comprising one, two, three or four heteroatoms selected from the group consisting of oxygen, nitrogen or halogens.

In a particular embodiment of the invention said R^{a} or R^{b} groups optionally comprise one carbonyl and/or carboxylic groups.

In the case where the substrate is a ketone or aldehyde, the corresponding alcohols (i.e (II-a)), are of formula

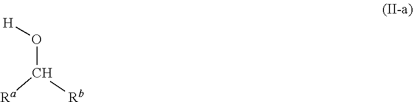

wherein R^{a} and R^{b} are defined as in formula (I).

In the case where the substrate is an ester/lactone, the corresponding alcohols (i.e (II-b) and (II-c)), or the corresponding diol (II-d), of said substrate (I), are of formula

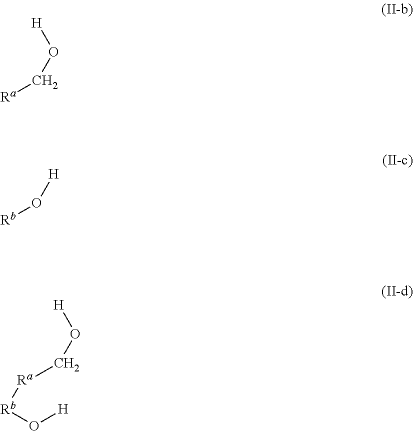

wherein R^{a} and R^{b} are defined as in formula (I).

A compound of formula (II-b or II-c) will be obtained in the case where n is 1 and R^{a} and R^{b} are not bonded together, while a compound of formula (II-d) will be obtained in the case where n is 1 and R^{a} and R^{b} are bonded together.

It is understood that said compounds (II-a) or (II-b)/(II-c)/(II-d) can be in a racemic or optically active form, depending on the nature of the substrate and on the catalyst/pre-catalyst used. According to a particular embodiment of the invention, the substrate (I) is a racemic or optically active compound.

It is understood that by “. . . hydrocarbon group . . . ” it is meant that said R^{a} or R^{b} can be in the form of a linear, branched or cyclic aromatic, alkyl, alkenyl, or alkynyl group, e.g., a linear alkyl group, or can also be in the form of a mixture of said type of groups, e.g. a specific R^{a} may comprise a linear alkyl, a branched alkenyl (e.g. having one or more carbon-carbon double bonds), a (poly)cyclic alkyl and an aryl moiety, unless a specific limitation to only one type is mentioned. Similarly, in all the below embodiments of the invention, when a group is mentioned as being in the form of more than one type of topology (e.g. linear, cyclic or branched) and/or unsaturation (e.g. alkyl, aromatic or alkenyl), it is meant also a group which may comprise moieties having any one of said topologies or unsaturations, as explained above. Similarly, in all the below embodiments of the invention, when a group is mentioned as being in the form of one type of unsaturation (e.g. alkyl), it is meant that said group can be in any type of topology (e.g. linear, cyclic or branched) or having several moieties with various topologies.

According to a further embodiment of the invention, the substrate is a ketone, an aldehyde or an ester, or a lactone, that will provide an alcohol, or a diol, that is useful in the pharmaceutical, agrochemical or perfumery industry as final product or as an intermediate. Particularly preferred substrates are ketones, aldehydes, esters, or lactones, that will provide an alcohol, or diol, which is useful in the perfumery industry as final product or as an intermediate.

According to another embodiment of the invention, the substrate is a C₅-C₂₀ compound of formula (I), and in particular one may cite those wherein R^{a} represent a hydrogen atom or a R^{b} group, R^{b} representing a linear, branched or cyclic C₁-C₂₀ hydrocarbon group optionally substituted and optionally comprising one, two or three oxygen or nitrogen atoms; or R^{a} and R^{b}, taken together, represent a C₃-C₂₀ hydrocarbon group, optionally substituted and optionally comprising one, two or three oxygen or nitrogen atoms.

According to a further embodiment of the invention, the substrate is a C₅-C₂₀ compound of formula (I), R^{a} represents a hydrogen atom or a R^{b} group, R^{b} representing a linear, branched or cyclic C₃-C₁₈ alkyl group optionally substituted, or a C₄-C₁₈ alkenyl or alkynyl group optionally substituted or a C₆-C₁₀ aromatic group optionally substituted; or R^{a} and R^{b}, taken together, represent a C₃-C₁₈ hydrocarbon group, optionally substituted.

Possible substituents of R^{a} and R^{b} are one, two or three halogen, OR^{c}, NR^{c}₂ or R^{c} groups, in which R^{c} is a hydrogen atom, a halogenated C₁-C₂ group or a C₁ to C₁₀ cyclic, linear or branched alkyl, or alkenyl group, preferably a C₁ to C₄ linear or branched alkyl or alkenyl group. As other possible substituents one may also cite a group COOR^{c}, which can also be reduced to the corresponding alcohol during the invention's process, according to the molar amount of H₂ used, as well known by a person skilled in the art.

According to a further embodiment of the invention, said substituents are one, two or three halogen, OR^{c}, or R^{c} groups, in which R^{c} is a hydrogen atom, or a C₁ to C₆ cyclic, linear or branched alkyl, or alkenyl group. As other possible substituents one may also cite a group COOR^{c}, which can also be reduced to the corresponding alcohol during the invention's process, according to the molar amount of H₂ used, as well known by a person skilled in the art.

Non-limiting examples of substrates of formula (I) are the following:
C₃₋₁₄ aldehydes such as:
a C₃₋₁₀ alkanal, a C₃₋₁₀ 2-alkenal, a C₃₋₁₀ 2-methyl-2-alkenal, a C₅₋₁₀ 2,4-dienal, a 3-alkyl-3-benzene-prop-2-enal, a 3-alkyl-2-methyl-3-benzene-prop-2-enal, a C₇₋₁₀-benzene-carbaldehyde, a C₄₋₁₂ 2-methylen-aldehyde;
wherein the underlined compounds are known to be particularly base-sensitive substrates; and
C₃₋₁₄ ketones such as:
a di(C₁-₁₂ alkyl)ketone, a C₄-C₁₂ cyclic-ketone, a cyclopentenone alpha substituted by a C₅-₁₂ hydrocarbon group, a cyclohexenone alpha substituted by a C₆₋₁₂ hydrocarbon group, a substituted aryl C₁₋₁₂-alkyl ketone, a C₂₋₁₂-1-alkene methyl ketone, a C₂₋₁₂-1-alkyne methyl ketone, a 2-trimethylsilyl-1-ethynyl C₁₋₁₀-alkyl ketone, 2-trimethylsilyl-1-ethynyl phenyl ketone, a 2-trimethylsilyl-1-ethynyl C₁₋₁₂-(un)substituted aryl ketone, a C₁₋₁₂-(un)substituted aryl chloromethyl ketone, a C₁₋₁₂-(un)substituted aryl chloromethyl ketone, a C₄₋₅-heteroaryl chloromethyl ketone, C₁₋₁₂-(un)substituted aryl dichloromethyl ketone, a C₁₋₁₂-alkyl dichloromethyl ketone, a C₄₋₅-heteroaryl dichloromethyl ketone, a C₁₋₁₂-(un)substituted aryl trichloromethyl ketone, a C₁₋₁₂-alkyl trichloromethyl ketone, a C₄₋₅-heteroaryl trichloromethyl ketone, a C₁₋₁₂-(un)substituted 1-indanone, a C₁₋₁₂-(un)substituted 1-tetralone, a C₁₋₁₂-(un)substituted 2-tetralone, a C₁₋₁₂-(un)substituted 1-benzosuberone, a C₁₋₁₂-(un)substituted 2-benzosuberone, a C₁₋₁₂-(un)substituted benzofuran-3(2H)-one, a C₁₋₁₂-(un)substituted 4-chromanone; and
wherein the underlined compounds are known to be particularly base-sensitive substrates, by, “C₁₋₁₂-(un)substituted” it is meant here a group such as an aryl(phenyl or naphthyl) which can be substituted by one or more groups which have in total between 1 and 12 carbon atoms; and
C₆₋₁₄ esters such as:
alkyl cinnamates, sorbates or salycilates, alkyl or glycolic esters of natural (fatty or not) acids, Sclareolide, spirolactones, allylic ester, di alkyl diesters, (un)substituted benzoic esters, and β-γ unsaturated esters. In particular, the substrate can be selected from the group consisting of sclareolide, C₉-C₁₅ spirolactones and C₁-C₄ alkyl esters of 4-methyl-6-(2,6,6-trimethyl-1-cyclohexen-1-yl)-3-hexenoic acid. One can also cite the di alkyl esters of 1,4-dicarboxylate-cyclohexane, the di C₁₋₅ alkyl esters of the C₂₋₁₀ alkanediyl-dicarboxylates, C₁₋₅ alkyl cyclopropanecarboxylates, mono-, di- or tri-methoxybenzoic esters.

According to a further embodiment of the invention, the substrate is a compound of formula

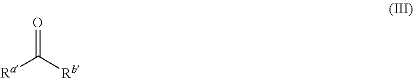

wherein R^{a′} represents a hydrogen atom or a C₁₋₄ alkyl or alkenyl group;
R^{b′} represents a C₅-C₁₄ hydrocarbon group, preferably alkyl or alkenyl, optionally substituted and optionally comprising one or two oxygen or nitrogen atoms; or
R^{a′} and R^{b′}, taken together, represent a C₄-C₁₆, hydrocarbon group, preferably alkyl, alkenyl or alkadienyl, optionally substituted and optionally substituted and optionally comprising one or two oxygen or nitrogen atoms.

According to a particular embodiment of the invention, the compound of formula (III) is a ketone.

Possible substituents of R^{a′} and R^{b′} are one or two OR^{c}, CONR^{c}₂ or R^{c} groups, in which R^{c} is a hydrogen atom or a C₁ to C₄ linear or branched alkyl or alkenyl group. As other possible substituents, one may also cite a group COOR^{c}, which can also be reduced to the corresponding alcohol during the invention's process, according to the molar amount of H₂ used, as well known by a person skilled in the art.

Non-limiting examples of substrates of formula (II) are the following:
C₁₁-C₁₈ ketones comprising a trimethyl-cyclohexyl or trimethyl-cyclohexenyl moiety, in particular the 2,6,6 trimethyl ones, such as: C₁-C₄ alkyl 2,6,6-trimethyl-4-oxo-2-cyclohexene-1-carboxylate, C₁-C₄ alkyl 4,6,6-trimethyl-2-oxo-3-cyclohexene-1-carboxylate, beta ionone or irone, 1-(2,2,3,6-tetramethyl-1-cyclohexyl)-1-hexen-3-one, 1-(2,2,6-trimethyl-1-cyclohexyl)-1-hexen-3-one or 4-acetyl-3,5,5-trimethyl-2-cyclohexen-1-one;
C₉-C₁₆ ketones comprising a 2,2,3-trimethyl-cyclopentenyl or 2,2,3-trimethyl-cyclopentyl moiety, such as: 4,4-dimethyl-6-(2,2,3-trimethyl-3-cyclopenten-1-yl)-5-hexen-3-one or 4,4-diethyl-6-(2,2,3-trimethyl-3-cyclopenten-1-yl)-5-hexen-3-one;
C₁₀-C16 ketones comprising a naphthalenone moiety, such as: 3,4,4a,5,8,8a-hexahydro-2,2,6,8-tetramethyl-1 (2H)-naphthalenone;
C₅-C₁₄ ketones comprising a cyclopentanone or cyclohexanone moiety, such as: 2-pentyl-1-cyclopentanone, 3,3,5-trimethylcyclohexanone, 2-ethyl-4,4-dimethyl-cyclohexanone, 2-tert-butylcyclohexanone or 4-tert-butylcyclohexanone; or
C₉-C18 ketones comprising a phenyl moiety, such as: 5-(3,4-dimethylphenyl)-2,2,4,5-tetramethyl-3-hexanone, 4-phenyl-3-buten-2-one, 4-phenyl-2-butanone, 1-phenyl-2-pentanone, 4-methyl-1-phenyl-2-pentanone or 2-methoxy-1-phenyl-1-ethanone; or
C₇-C₁₂ acyclic ketones, such as: 1-octen-3-one.

In the present invention, contrary to almost all the examples in the prior art, the presence of a base is not mandatory. This is an advantage, since it allows significant increases in yields for the production of alcohols from base-sensitive substrates. Therefore, according to a particular embodiment of the invention, the substrate is a base-sensitive compound and the process is carried out in the absence of a base.

According to any one of the above embodiments of the invention, particularly suitable substrates as those of formula (I) wherein n is 0, i.e. aldehydes or ketones, and in particular ketones.

According to a particular embodiment of the invention, the ruthenium catalyst or pre-catalyst (also referred to from herein as complex) can be of the general formula

[Ru(P—PO)(N—N)(S)₂₋ᵣYᵣ](Z)₂₋ᵣ (A)
wherein r represents 0, 1 or 2;
S represents a neutral C₁-C₂₆ neutral monodentate ligand;
(P—PO) represents a ligand as defined above;
(N—N) represents a ligand as defined above; and
each Y represents, simultaneously or independently, a hydrogen atom, a hydroxyl, a C₁-C₁₀ alkoxyl, a halogen atom (such as Cl, Br or I), or an C₃-C₁₅ allyl group; and
each Z represents, simultaneously or independently, ClO₄⁻, BF₄—, PF₆—, SbCl₆—, AsCl₆—, SbF₆—, AsF₆—, a R^{d}SO₃⁻ wherein R^{d} is a chlorine of fluoride atom or an C₁-C₈ alkyl, aryl, fluoroalkyl or fluoroaryl group, or a BR^{e}₄⁻ wherein R^{e} is a phenyl group optionally substituted by one to five groups such as halide atoms and/or methyl and/or CF₃ groups.

The monodentate ligands can be a phosphine, like PPh₃, CO or even a solvent. By the term “solvent” it has to be understood the usual meaning in the art and in particular compounds used as diluent in the preparation of the complex or during the invention's process. Non limiting examples of such solvent are dimethylsulfoxide, acetonitrile, dimethylformamide, an alcohol (e.g. an C₁-C₄ alcohol), or also THF, acetone, pyridine or a C₃-C₈ ester or the substrate of the invention's process.

In a particular embodiment of the invention, in formula (A), each Y represents, simultaneously or independently, a hydrogen atom, a hydroxyl, a C₁ to C₁₀ alkoxyl group, such as a methoxyl, ethoxyl or isopropoxyl group, a halogen atom (such as Cl, Br or I), or a C₃-C₆ allyl group group, such as allyl (i.e. propenyl), 2-methyl-allyl (i.e. 2-methyl-propenyl).

In a particular embodiment of the invention, in formula (A), each Z represents, simultaneously or independently, ClO₄⁻, BF₄—, PF₆—, SbCl₆—, AsCl₆—, SbF₆—, AsF₆—, a R^{d}SO₃⁻ wherein R^{d} is a chlorine of fluoride atom or a CF₃ group, or a BR^{e}₄⁻ wherein R^{e} is a phenyl group optionally substituted by one, two or three groups such as halide atoms and/or methyl and/or CF₃ groups.

According to a particular embodiment of the invention, there can be used as complex a compound of formula

[Ru(P—PO)(N—N)Y₂] (A′)

wherein P—PO, N—N and Y have the meaning indicated above.

The complexes of the invention can be added into the reaction medium of the invention's process in a large range of concentrations. As non-limiting examples, one can cite as complex concentration values those ranging from 50 ppm to 50000 ppm, relative to the amount of substrate. Preferably, the complex concentration will be comprised between 100 and 10000, or even 1000, ppm. It goes without saying that the optimum concentration of complex will depend, as the person skilled in the art knows, on the nature of the latter, on the nature of the substrate and on the pressure of H₂ used during the process, as well as the desired time of reaction.

The hydrogenation processes of the invention may be carried out in the absence of a base, or in the absence of a significant amount of a base, or in the presence of a base. The base can be added in particular for those processes wherein neither the starting product nor the final product is sensible to a base (e.g. does not undergo isomerisations, degradation, ring opening, polymerisations reactions, etc). Said base can be the substrate itself, if the latter is basic, a corresponding alcoholate or any organic or inorganic base having preferentially a pKₐ above 10. According to a particular embodiment of the invention, said base may have a pKₐ above 14. It is also understood that preferably said base does not reduce itself a substrate of formula (I). As non-limiting examples one may cite the following type of base: C₁₋₈ alcoholate, hydroxides, alkaline or alkaline-earth carbonates, C₁₀₋₂₆ phosphazenes, C₁₋₁₀ amides, basic alox, siliconates (i.e. silicium derivatives having SiO⁻ or SiRO⁻ groups), or an inorganic hydrides such as NaBH₄, NaH or KH.

One can cite, as non-limiting examples, alkaline or alkaline-earth metal carbonates, such as cesium carbonate, an alkaline or alkaline-earth metal hydroxide, C₁₋₁₀ amidures, C₁₀₋₂₆ phosphazene or an alcoholate of formula (R³¹O)₂M or R³¹OM′, wherein M is an alkaline-earth metal, M′ is an alkaline metal or an ammonium NR³²₄⁺, R³¹ stands for hydrogen or a C₁ to C₆ linear or branched alkyl radical and R³² stands for a C₁ to C₁₀ linear or branched alkyl radical, such as sodium or potassium alcoholates. Of course, other suitable bases can be used.

According to an embodiment of the invention, said base is an alkaline alcoholate of formula R³¹OM′.

Useful quantities of base, added to the reaction mixture, may be comprised in a relatively large range. One can cite, as non-limiting examples, ranges between 0 to 50000 molar equivalents, relative to the complex (e.g. base/com=up to 50000), preferably 0 to 2000, and even more preferably between 2 and 1000 molar equivalents.

The hydrogenation reaction can be carried out in the presence or absence of a solvent. When a solvent is required or used for practical reasons, then any solvent current in hydrogenation reactions can be used for the purposes of the invention. Non-limiting examples include C₆₋₁₀ aromatic solvents such as toluene or xylene, C₅₋₈ hydrocarbon solvents such as hexane or cyclohexane, C₃₋₉ ethers such as tetrahydrofuran or MTBE, polar solvents such as C₂₋₅ primary or secondary alcohols such as isopropanol or ethanol, or mixtures thereof. The choice of the solvent is a function of the nature of the complex and the person skilled in the art is well able to select the solvent most convenient in each case to optimize the hydrogenation reaction.

In the hydrogenation process of the invention, the reaction can be carried out at a H₂ pressure comprised between 10⁵ Pa and 80×10⁵ Pa (1 to 80 bars) or even more if desired. Again, a person skilled in the art is well able to adjust the pressure as a function of the catalyst load and of the dilution of the substrate in the solvent. As examples, one can cite typical pressures of 1 to 50×10⁵ Pa (1 to 50 bar).

The temperature at which the hydrogenation can be carried out is comprised between −20° C. and 120° C. More preferably in the range of between 0° C. and 100° C. Of course, a person skilled in the art is also able to select the preferred temperature as a function of the melting and boiling point of the starting and final products as well as the desired time of reaction or conversion.

According to any of the embodiments of the present invention, the diamino bidentate ligand (N—N) can be a racemic or an optically active compound of formula

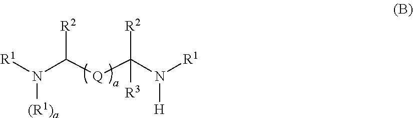

wherein each a, simultaneously or independently, represents 0 or 1;
the R¹, taken separately, represent, simultaneously or independently, a hydrogen atom or a C₁₋₁₀ alkyl or alkenyl group optionally substituted; two R¹, taken together, may form a saturated heterocycle containing 5 to 10 atoms and including the atoms to which said R¹ are bonded, said heterocycle being optionally substituted;
R² and R³, taken separately, represent, simultaneously or independently, a hydrogen atom, a C₁₋₁₀ alkyl or alkenyl group optionally substituted or a C₆₋₁₀ aromatic group optionally substituted; a R¹ and an adjacent R², taken together, may form a saturated or aromatic heterocycle containing 5 to 12 atoms and including the atoms to which said R¹ and R² are bonded, and being optionally substituted and optionally containing one additional nitrogen or oxygen atoms, or also sulfur atom; two R², or a R² and a R³, taken together, may form a saturated or unsaturated ring having 5 to 12 atoms and including the carbon atom to which said R² or R³ groups are bonded, said ring being optionally substituted and optionally containing one additional nitrogen and/or oxygen and/or sulfur atoms; and
Q represents a
   a group of formula

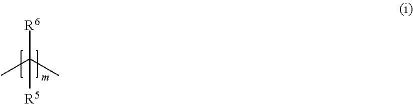

wherein m is 1 or 2, and
R⁵ and R⁶ represent, simultaneously or independently, a hydrogen atom, a C₁₋₁₀ alkyl or alkenyl group optionally substituted, a C₆₋₁₀ aromatic group optionally substituted, or an OR⁷ group, R⁷ being a C₁₋₁₀ alkyl or alkenyl group; two distinct R⁶ and/or R⁵ groups, or R⁵ or R⁶ and R¹ or R², taken together, may form a C₃₋₈, or even up to C₁₀, saturated or unsaturated ring optionally substituted, including the atoms to which said R⁶, R⁵, R¹ and/or R² groups are bonded, and optionally containing one or two additional nitrogen, oxygen or sulfur atoms; or
a C₁₀-C₁₆ metallocenediyl, a 2,2′-diphenyl, a 1,1′-binaphthalene-2,2′-diyl, a benzenediyl, a naphthalenediyl, a 4,12-[2:2]-paracyclophanediyl, a 1,6-spiro[4:4]nonanediyl, 3,4-(1-benzyl)-pyrrolidinediyl, 2,3-bicyclo[2:2:1 ]hept-5-enediyl, 4,6-phenoxazinediyl, 4,5-(9,9-dimethyl)-xanthenediyl, 3,3′-bipyri-4,4′-diyl or 2,2′-(1,1′-bicyclopentyl)-diyl group optionally substituted.

According to an embodiment, by “aromatic group or ring” it is meant a phenyl or naphthyl group.

As mentioned above, in said ligand (B) the atoms which may coordinate the Ru atom are the two N atoms bearing the R¹ groups. Therefore, it is also understood that whenever said R¹, R², R³, R⁵, R⁶ or any other group comprises heteroatoms such as N, O or S, said heteroatoms are not coordinating.

Possible optional substituents of R¹, R², R³, R⁵, R⁶ or Q are one, two, three or four groups selected amongst i) halogens (in particular when said substituents are on aromatic moieties), ii) C₅₋₁₂ cycloalkyl or cycloalkenyl, iii) C₁₋₁₀ alkoxy, alkyl, alkenyl, polyalkyleneglycols or halo- or perhalo-hydrocarbon, iv) COOR⁴ wherein R⁴ is a C₁₋₆ alkyl, or v) a benzyl group or a fused or non-fused phenyl or indanyl group, said group being optionally substituted by one, two or three halogen, C₁₋₈ alkyl, alkoxy, amino, nitro, ester, sulfonate or halo- or perhalo-hydrocarbon groups. The Q group may also be substituted by one or two groups of formula O—(CR⁸₂)ₙ—O or O—(CR⁸₂)ₙ—NR⁴ wherein n is 1 or 2 and R⁸ being a hydrogen atom or a C₁₋₄ alkyl group. The expression “halo- or perhalo-hydrocarbon” has here the usual meaning in the art, e.g. a groups such as CF₃ or CClH₂ for instance.

For the sake of clarity, and as mentioned above, in any one of the embodiment of the present invention, whenever two groups of formula (B) are taken together to form a cycle or ring said cycle or ring can be a mono or bi-cyclic group.

According to a particular embodiment said compound (B) is one wherein each a, simultaneously or independently, represents 0 or 1;
the R¹, taken separately, represents, simultaneously or independently, a hydrogen atom or a C₁₋₆ alkyl or alkenyl group optionally substituted; two R¹, taken together, may form a saturated heterocycle containing 5 to 10 atoms and including the atoms to which said R¹ are bonded, said heterocycle being optionally substituted;
R² and R³, taken separately, represent, simultaneously or independently, a hydrogen atom, a C₁₋₆ alkyl or alkenyl group optionally substituted or a phenyl group optionally substituted; a R¹ and an adjacent R², taken together, may form a saturated heterocycle containing 5 or 10 atoms and including the atoms to which said R¹ and R² are bonded, and being optionally substituted and optionally containing one additional nitrogen or oxygen atoms; or R¹ and an adjacent R², taken together, may form a pyridinyl group including the atoms to which said R¹ and R² are bonded, and being optionally substituted; two R², or a R² and a R³, taken together, may form a saturated or unsaturated ring having 5 or 6 atoms and including the atoms to which said R² or R³ groups are bonded, said ring being optionally substituted and optionally containing one additional oxygen atoms; and
Q represents a
   a group of formula

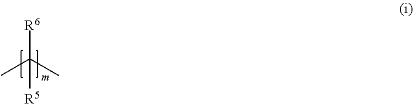

wherein m is 1 or 2, and
R⁵ and R⁶ represent, simultaneously or independently, a hydrogen atom, a C₁₋₆ alkyl or alkenyl group optionally substituted or a phenyl group optionally substituted; two distinct R⁶ and/or R⁵ groups, or R⁶ or R⁵ and a R¹ or R², taken together, may form a C_{3-6,} saturated or unsaturated ring optionally substituted, including the atoms to which said R⁶, R⁵, R¹ and/or R² groups are bonded and optionally containing one or two additional oxygen atoms.

Possible substituents of R¹, R², R³, R⁵, R⁶ or Q are one, two or three groups selected amongst i) halogens (in particular when said substituents are on aromatic moieties), ii) C₅₋₆ cycloalkyl or cycloalkenyl, iii) C₁₋₆ alkoxy or alkyl, iv) COOR⁴ wherein R⁴ is a C₁₋₄ alkyl, or v) a benzyl group or a fused or non-fused phenyl or indanyl group, said group being optionally substituted by one, two or three halogen, C₁₋₄ alkyl, alkoxy, amino, nitro, ester or sulfonate groups. The Q group may also be substituted by one or two groups of formula O—(CR⁸₂)ₙ—O, n being for 2 and R⁸ being a hydrogen atom or a methyl or ethyl group group. By “halo- or perhalo-hydrocarbon” it is meant groups such as CF₃ or CClH₂ for instance.

According to any embodiments of the present invention, a particular embodiment of formula (B) is represented by formula

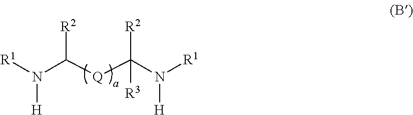

wherein a represents 0 or 1; and
each R¹, simultaneously or independently, represents a hydrogen atom or a C₁₋₄ alkyl group optionally substituted;
R² and R³, taken separately, represent, simultaneously or independently, a hydrogen atom, a C₁₋₄ alkyl group optionally substituted or a phenyl group optionally substituted; a R¹ and an adjacent R², taken together, may form a saturated heterocycle containing 5 or 6 atoms and including the atoms to which said R¹ and R² are bonded, and being optionally substituted and optionally containing one additional nitrogen or oxygen atoms; two R², or a R² and a R³, taken together, may form a saturated or unsaturated ring having 5 or 6 atoms and including the atoms to which said R² or R³ groups are bonded, said ring being optionally substituted and optionally containing one additional oxygen atom; and
Q represents a
   a group of formula

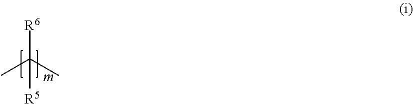

wherein m is 1 or 2, and
R⁵ and R⁶ represent, simultaneously or independently, a hydrogen atom, a C₁₋₄ alkyl group optionally substituted or a phenyl group optionally substituted; two distinct R⁶ and/or R⁵ groups, or R⁶ or R⁵ and a R¹ or R², taken together, may form a C₃₋₆, saturated or unsaturated ring optionally substituted, including the atoms to which said R⁶, R⁵, R¹ and/or R² groups are bonded and optionally containing one or two additional oxygen atoms.

According to a particular embodiment of the invention, said Q can be a group of formula (i) wherein m is 1 or 2, R⁵ is a hydrogen atom and R⁶ is as defined above.

Alternatively, according to any embodiments of the present invention, a particular embodiment of formula (B) is represented by formula

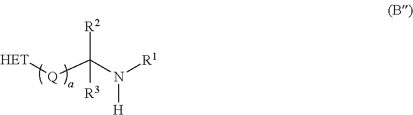

wherein a represents 0 or 1; and
R¹ represents a hydrogen atom or a C₁₋₄ alkyl group optionally substituted;
R² and R³, taken separately, represent, simultaneously or independently, a hydrogen atom, a C₁₋₄ alkyl group optionally substituted or a phenyl group optionally substituted; R¹ and R², or R² and R³, taken together, may form a saturated cycle containing 5 or 6 atoms and including the atoms to which said R¹, R² or R³ are bonded, and being optionally substituted and optionally containing one additional nitrogen and/or oxygen atom; and
HET represents a 2-pyridinyl group optionally substituted by one, two or three C₁₋₄ alkyl groups or by a benzyl group or a fused or non-fused phenyl or indanyl group, said group being optionally substituted by one, two or three halogen, C₁₋₄ alkyl, alkoxy, amino, nitro, ester or sulfonate groups; such as a 2-pyridyl, 2-quinolinyl or a methyl-2-pyridinyl; and
Q represents a
   a group of formula

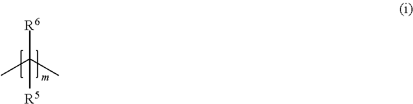

wherein m is 1 or 2, and
R⁵ and R⁶ represent, simultaneously or independently, a hydrogen atom, a C₁₋₄ alkyl group optionally substituted or a phenyl group optionally substituted; two distinct R⁶ and/or R⁵ groups, or R⁶ or R⁵ and a R¹ or R² or R⁹ or R^{9′}, taken together, may form a C₃₋₆, saturated or unsaturated ring optionally substituted, including the atoms to which said R⁶, R⁵, R¹ and/or R² groups are bonded and optionally containing one or two additional oxygen atoms.

Possible substituents of R¹, R², R³, R⁵, R⁶ or Q of formulae (B′) or (B″) are one or two i) halogens (in particular when said substituents are on aromatic moieties), ii) C₁₋₅ alkyl or alkoxy groups, iii) COOR^{f} wherein R^{f} is a C₁₋₄ alkyl, or iv) a benzyl group or a fused or non-fused phenyl group, said group being optionally substituted by one, two or three halogen, C₁-₄ alkyl or alkoxy groups, esters or sulfonate groups.

According to any one of the above-mentioned embodiments, at least one coordinating amino group of the N—N ligand is a primary amino groups (i.e. NH₂) or in other words in formula (B), (B′) or (B″) one, two or three R¹ represent a hydrogen atom.

According to any one of the above-mentioned embodiments, the coordinating amino group of the N—N ligand is a primary amino group (i.e. NH₂) or in other words in formula (B) or (B′) all R¹ represent a hydrogen atom.

According to any one of the above-mentioned embodiments, the N—N ligand is of n formula (B′), and preferably the two R¹ represent a hydrogen atom.

As non limiting examples of N—N ligands, one can cite the ones in the following Scheme (A):

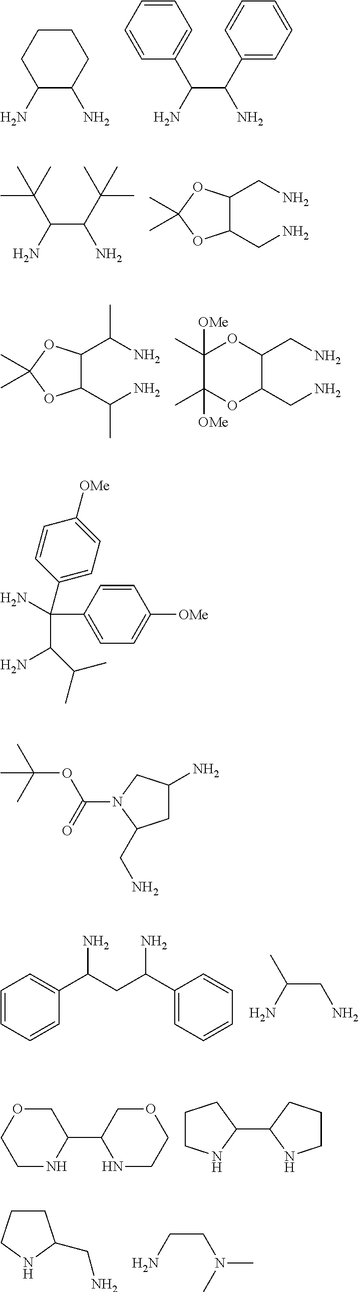

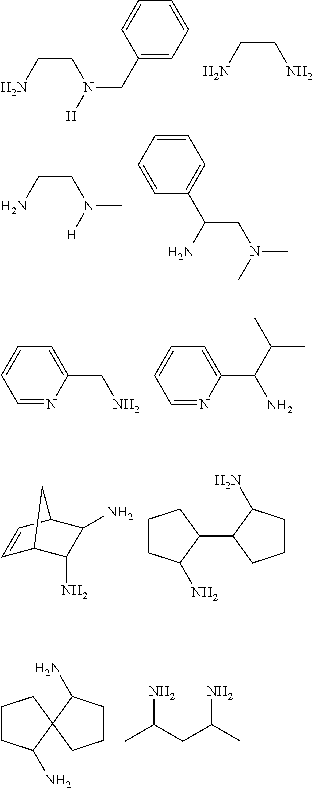

said compounds being in an optically active form or in a racemic form, if applicable.

According to any of the embodiments of the present invention, the bidentate ligand (P—PO) can be a racemic or an optically active compound of formula

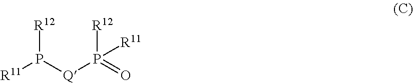

wherein R¹¹ and R¹², when taken separately, represent, simultaneously or independently, a C₁₋₈ alkyl or alkenyl group optionally substituted or a C₆₋₁₀ aromatic group optionally substituted; or the R¹¹ and R¹² bounded to the same phosphorous atom, when taken together, may form a saturated or unsaturated ring optionally substituted, having 4 to 8 atoms and including the phosphorus atom to which said R¹¹ and R¹² groups are bonded; and
Q′ represents
a group of formula

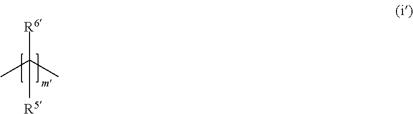

wherein m′ is 1, 2, 3 or 4 and
R^{5′} and R^{6′} represent, simultaneously or independently, a hydrogen atom, a C₁₋₁₀ alkyl or alkenyl group optionally substituted or a C₆₋₁₀ aromatic group optionally substituted, or an OR^{7′} group, R^{7′} being a linear, branched or cyclic C₁₋₁₀ alkyl or alkenyl group; two distinct R^{6′} and/or R^{5′} groups, taken together, may form a C₃ to C_{8,} or even up to C₁₀, saturated or unsaturated ring optionally substituted, including the atoms to which said R^{6′} and/or R^{5′} groups are bonded, and optionally containing one or two additional nitrogen or oxygen atoms; or
a C₁₀-C₁₆ metallocenediyl, a 2,2′-diphenyl, a 1,1′-binaphthalene-2,2′-diyl, a benzenediyl, a naphthalenediyl, a 4,12-[2:2]-paracyclophanediyl, a 1,6-spiro[4:4]nonanediyl, 3,4-(1-benzyl)-pyrrolidinediyl, 2,3-bicyclo[2:2:1]hept-5-enediyl, 4,6-phenoxazinediyl, 4,5-(9,9-dimethyl)-xanthenediyl, 3,3′-bipyri-4,4′-diyl or 2,2′-(1,1′-bicyclopentyl)-diyl group optionally substituted.

As mentioned above, according to a particular embodiment of the invention, by “aromatic group or ring” for (P—PO) it is also meant a phenyl or naphthyl derivative.

As mentioned above, in said ligand (C) the atoms which may coordinate the Ru atom are the P atoms of the PR¹¹R¹² group and the O atom of the POR¹¹R¹² groups. Therefore, it is also understood that whenever said R^{5′}, R^{6′}, R¹¹, R¹², Q′ or any other group comprises heteroatoms such as N or O, said heteroatoms are not coordinating.

Possible substituents of R^{5′}, R^{6′}, R¹¹ and R¹² are one to five halogens (in particular when said substituents are on aromatic moieties), or one, two or three i) C₁₋₁₀ alkyl alkenyl, alkoxy, polyalkyleneglycols groups or halo- or perhalo-hydrocarbon, amine or quaternary amine groups, ii) COOR^{h} wherein R^{h} is a C₁₋₆ alkyl group, iii) C₅₋₁₂ cycloalkyl or cycloalkenyl group, iv) NO₂ group, or v) a benzyl group or a fused or non-fused phenyl, indanyl or naphthyl group, said group being optionally substituted by one, two or three halogen, C₁₋₈ alkyl, alkoxy, amino, nitro, ester, sulfonate or halo- or perhalo-hydrocarbon groups. By “halo- or perhalo-hydrocarbon” it is meant groups such as CF₃ or CClH₂ for instance.

The Q′ group may be also substituted by one or two groups of formula O—(CR^{8′}₂)_{n′}—O or O—(CR^{8′}₂)_{n′}—NR^{4′} wherein n′ is 1 or 2, R^{4′} being a C₁₋₄ alkyl group and R^{8′} being a hydrogen atom or a C₁₋₄ alkyl group.

For the sake of clarity, and as mentioned above, in any one of the embodiment of the present invention, whenever two groups of formula (C) are taken together to form a cycle or ring, said cycle or ring can be a mono or bi-cyclic group.

In a particular embodiment of formula (C), P—PO is a bidentate ligand wherein R¹¹ and R¹² represent, simultaneously or independently, a C₁₋₆ alkyl group optionally substituted, a phenyl or naphthyl group optionally substituted; or the groups R¹¹ and R¹² bounded to the same phosphorous atom, taken together, form a saturated or unsaturated ring optionally substituted, having 5 to 7 atoms and including the phosphorus atom to which said R¹¹ and R¹² groups are bonded;
Q′ represents:
   a group of formula (i′) wherein m′ is 1, 2 or 3, and
   R^{5′} and R^{6′} represent, simultaneously or independently, a hydrogen atom, a C₁₋₄ alkyl group optionally substituted or a phenyl group optionally substituted; two distinct R^{6′} and/or R^{5′} groups, taken together, may form a C₃₋₆, saturated or unsaturated ring optionally substituted, including the atoms to which said R^{6′} and/or R^{5′} groups are bonded and optionally containing one or two additional oxygen atoms; or
   a C₁₀-C₁₂ ferrocenediyl, a 2,2′-diphenyl, a 1,1′-binaphthalene-2,2′-diyl, a benzenediyl, a naphthalenediyl, a 4,12-[2:2]-paracyclophanediyl, a 1,6-spiro[4:4]nonanediyl, 3,4-(1-benzyl)-pyrrolidinediyl, 2,3-bicyclo[2:2:1]hept-5-enediyl, 4,6-phenoxazinediyl, 4,5-(9,9-dimethyl)-xanthenediyl, 3,3′-bipyri-4,4′-diyl or 2,2′-(1,1′-bicyclopentyl)-diyl group optionally substituted.

Possible substituents of R⁵′, R⁶′, R¹¹ and R¹² are one to five halogens (in particular when said substituents are on aromatic moieties), or one, two or three i) C₁₋₆ alkyl alkenyl, alkoxy or amine groups, ii) COOR^{h} wherein R^{h} is a C₁₋₆ alkyl group, iii) C₅₋₆ cycloalkyl or cycloalkenyl group, iv) NO₂ group or v) a benzyl group or a fused or non-fused phenyl or naphthyl group, said group being optionally substituted by one, two or three halogen, C₁₋₆ alkyl, alkoxy, amino, nitro, ester, sulfonate or halo- or perhalo-hydrocarbon groups.

The Q′ group may also be substituted by one or two groups of formula O—(CR^{8′}₂)_{n′}—O or O—(CR^{8′}₂)_{n′}—NR^{4′} wherein n′ is 1 or 2, R^{4′} being a C₁₋₄ alkyl group and R^{8′} being a hydrogen atom or a C₁₋₄ alkyl group.

According to any embodiment of the invention, for this P—PO ligands, Q′ may represent a linear C₁₋₅ alkanediyl radical optionally substituted, a 1,2- or 1,1′-C₁₀₋₁₂ metallocenediyl, a 2,2′-diphenyl, a 1,1′-(bis(naphthyl)-2,2′-diyl, a 1,2-benzenediyl or a 1,8- or 1,2-naphthalenediyl group optionally substituted.

According to a particular embodiment of the invention, said P—PO ligand is a compound of formula (C) wherein
R¹¹ and R¹² represent, simultaneously or independently, a phenyl group optionally substituted; or the groups R¹¹ and R¹² bounded to the same phosphorous atom, taken together, form a saturated ring optionally substituted, having 4 to 7 atoms and including the phosphorus atom to which said R¹¹ and R¹² groups are bonded; and
Q′ represents a C₂-C₄ alkanediyl radical optionally substituted, a C₁₀-C₁₂ ferrocenediyl, a 2,2′-diphenyl, a 1,1′-(bis(naphthyl)-2,2′-diyl, a 1,2-benzenediyl or a naphthalenediyl group optionally substituted.

Possible substituents of said R¹¹, R¹² or Q′ are as described above.

Furthermore, in all the above embodiments, a particularly appreciated mode of realisation is the one where said R¹¹ and R¹² groups are aromatic groups optionally substituted.

As non limiting examples of P—PO ligands, one can cite the ones in the following Scheme (B):

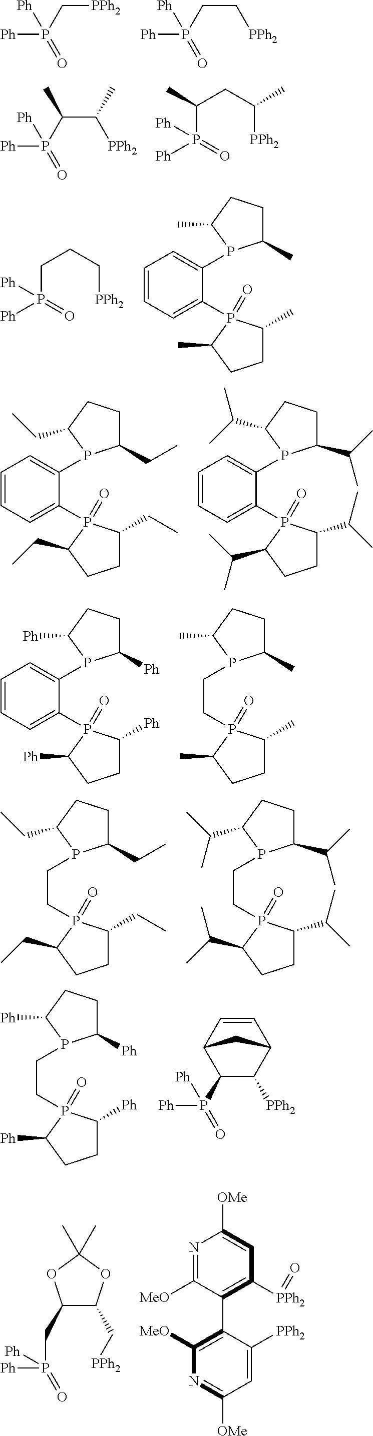

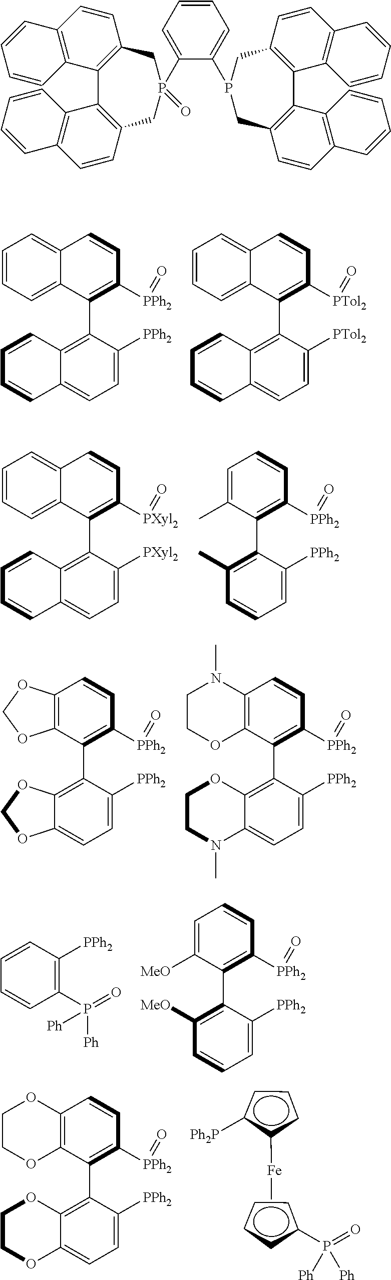

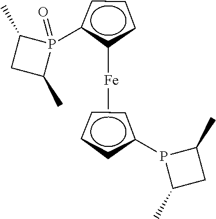

said compounds being in an optically active form or in a racemic form, if applicable.

The ligands (B) or (C) described above can be obtained by applying standard general methods which are well known in the state of the art and by the person skilled in the art. Many of said ligands N—N or P—PO are even commercially available.

The complexes of formula (A) or (A′), as above described, as well as those wherein Y is also a C₁-C₈ acyloxyl or a halogen atom (such as Cl, Br or I), which are precursors (as below described) of said complexes (A) or (A′), are new compounds and are therefore another object of the present invention.

The complex (A) of the invention can be used in the form of a preformed complex or can be generated in situ, in the reaction medium of the hydrogenation.

In any case, according to a particular embodiment of the invention, the catalyst or pre-catalyst is obtained or obtainable by a process comprising reacting together:
1) a ruthenium precursor of formula

[Ru(“diene”)(L)ᵥE₂₋ᵥ] (D)
wherein v represents 0, 1 or 2;
E represents a mono anion;
“diene” represents a linear or branched C₄-C₁₅ hydrocarbon group comprising two carbon-carbon double bonds, optionally substituted, or a cyclic C₇-C₂₀ hydrocarbon group comprising two carbon-carbon double bonds, optionally substituted; and
L represents a C₃-C₁₅ allyl, a C₆-C₁₂ aromatic ring optionally substituted or a C₇-C₁₅ triene;
2) with a C₂₋₄₀ diamino bidentate ligand (N—N), defined as above, and a C₆₋₅₀ bidentate ligand (P—PO), defined as above; and
3) with optionally between approximately 0.5 and 2.1 molar equivalent of base.

Optional substituent of the “diene” or of L are one or two C₁-C₁₀ alkyl or aryl groups, C₁-C₆ alkoxy groups or —C(O)O—(C₁-C₆ alkyl) groups.

It is understood that “allyl” possesses the usual meaning in the art, i.e. a group comprising a fragment C═C—C.., or C═C-C.. Similarly, it is understood that “triene” possesses the usual meaning in the art, i.e. a group comprising three non aromatic carbon-carbon double bonds.

According to a particular embodiment of the invention, E represents a mono anion selected amongst the group consisting of halides (e.g. Cl, Br, I,), BF₄—, ClO₄⁻, PF₆—, SbCl₆—, AsCl₆—, SbF₆—, AsF₆—, hydroxylate, C₁-C₁₀ carboxylates (e.g. acetate, trifluoroacetate, proprionate, 2-Et-hexanoate), a C₅-C₁₀ 1,3-diketonate, RⁱSO₃⁻ wherein Rⁱ is a chlorine of fluoride atom or a C₁-C₈ alkyl, aryl, fluoroalkyl or fluoroaryl group, or BR^{j}₄⁻ wherein R^{j} is a phenyl group optionally substituted by one to five groups such as halide atoms or methyl or CF₃ groups.

As non-limiting examples of suitable ruthenium precursors one can cite the compound (D) wherein “diene” stands for a C₇-C₁₀, hydrocarbon group comprising two carbon-carbon double bonds, such as for example COD (cycloocta-1,5-diene) or NBD (norbornadiene), or yet cyclohepta-1,4-diene.

As non-limiting examples of suitable ruthenium precursors, one can cite the compound (D) wherein “allyl” stands for a C₃-C₁₀, or even C₃-C₆, hydrocarbon group comprising a fragment C═C—C.., or C═C—C., such as for example allyl or 2-methyl-allyl (see, for instance, J. Powell et al., in J. Chem. Soc., (A), 1968, 159; M. O. Albers et al., Inorganic Synth., 1989, 26, 249; R. R. Schrock et al., J. Chem. Soc. Dalton Trans., 1974, 951).

As non-limiting examples of suitable ruthenium precursors, one can cite the compound (D) wherein “aromatic ring” stands for a C₆-C₁₂ group comprising a benzene ring, such as an indane or a p-cymene such as for example benzene, para-cymene (6-isopropyl-toluene) or hexamethyl benzene.

As non-limiting examples of suitable ruthenium precursors, one can cite the compound (D) wherein “triene” stands for a C₇-C₁₀ hydrocarbon group comprising three non aromatic carbon-carbon double bonds, such as for example COT (cyclooctatriene).

The preparation of the catalyst may benefit from the presence of a base, in particular when in compound (D) E represents a halogen or a carboxylate group. Said base can be defined as for the base of the hydrogenation process described herein above.

As specific, but non limiting, examples of said ruthenium precursor (D), one may cite the following:
[Ru(“diene”)(“allyl”)₂] such as [Ru(COD)(2-methallyl)₂], [Ru(COD)(allyl)₂], [Ru(NBD)(2-methallyl)₂] or [Ru(NBD)(allyl)₂];
[Ru(“diene”)E₂] such as [Ru(COD)(Cl)₂] or [Ru(NBD)(Cl)₂];
[Ru(“diene”)(“triene”)] such as [Ru(COD)(COT)]; or
[Ru(“diene”) (“arene”)] such as [Ru(COD)(C₆H₆)], [Ru(C₆H₆)(cyclohexadiene)], [Ru(COD)(C₁₀H₈)], [Ru(COD)(1,4-C₆H₄Me₂)] or [Ru(COD)(1,3,5-C₆H₃Me₃)].

The preparation of the catalyst/pre-catalyst can be carried out in a suitable solvent. As a person skilled in the art is well aware, the solvent is preferably anhydrous, e.g. containing less that 0.1% of water. Said solvent could be the substrate of the hydrogenation processes itself or another one. Typically there is used the same solvent as for the subsequent hydrogenation as described herein above. Typical non-limiting examples are given herein below, when describing the hydrogenation process.

A typical example of such procedure to prepare the invention's catalysts is provided in the examples.

Alternatively, said catalyst or pre-catalyst can be obtained by a process comprising reacting together a complex of formula [Ru(P—PO)(Arene)(Y)₂] or [Ru(P—PO)(Arene)Y]Y with a (N—N) ligand. Typical examples for the synthesis of complexes of formula [Ru(P—PO)(Arene)Y]Y is provided in J. W. Faller, B. J. Grimmond, D. J. D'Alliessi J. Am. Chem. Soc. 2001, 123, 2525-2529.

### EXAMPLES

The invention will now be described in further detail by way of the following examples, wherein the temperatures are indicated in degrees centigrade and the abbreviations have the usual meaning in the art.

All the procedures described hereafter have been carried out under an inert atmosphere unless stated otherwise. Hydrogenations were carried out in open glass tubes placed inside a stainless steel autoclave. H₂ gas (99.99990%) was used as received. All substrates and solvents were distilled from appropriate drying agents under Ar. NMR spectra were recorded on a Bruker AM-400 (¹H at 400.1 MHz, ¹³C at 100.6 MHz, and ³¹P at 161.9 MHz) spectrometer and normally measured at 300 K, in CDCl₃ unless indicated otherwise. Chemical shifts are listed in ppm.

### Example 1

Catalytic hydrogenation of acetophenone using various invention ruthenium complexes: formation in situ, from various Ru precursor with (phosphine-phosphinoxide ligands (La-Ld) as (P—PO) and diamines (Lf-Lg) as (N—N), the hydrogenation process carried out in the absence or presence of a base.

A typical experimental procedure is as follows:

In a glove box under argon, a solution of acetophenone (2.404 g, 20 mmol) and n-tridecane (187.8 mg, 1.02 mmol) in iPrOH (2 ml) were added, followed by more iPrOH (2×1 ml), to a Keim autoclave equipped with a magnetic stirring bar and containing the precursor (e.g. Ru(COD)(C₄H₇)₂) (0.02 mmol, 0.1 mol %), the phosphine-phosphinoxide (0.022 mmol, 0.11 mol %, see Table a), (1R,2R)-1,2-diphenylethylenediamine (4.7 mg, 0.022 mmol, 0.11 mol %) and iPrOH (6 ml). The autoclave was pressurized with hydrogen gas at 50 bar and placed in a thermostated oil bath set at 60° C. After the indicated time, the autoclave was removed from the oil bath, and cooled in a cold-water bath. The autoclave was vented and opened; an aliquot (0.4 ml) was taken, diluted with MTBE (5 ml), filtered over a plug of celite® 560 and analyzed by GC (DB-Wax). Under these conditions several phosphine-phosphinoxides (Table 1) and diamines (Table 2) were tested, as reported in Table 3.

TABLE 1
Structure of phosphine-phosphinoxides ligands (La-Ld) used
Structure 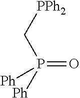
La 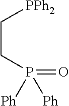
Lb 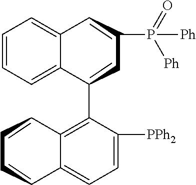
Lc 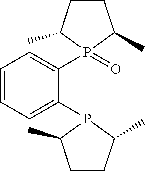
Ld 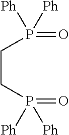
Le (not part of the invention)

wherein Ph is a C₆H₅ group.

TABLE 2
Structure of diamines ligands (Le-Lg) used
Structure 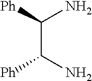
Lf 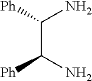
Lg 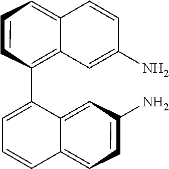
Lf (not part of the invention)

wherein Ph is a C₆H₅ group.

TABLE 3
Hydrogenation of acetophenone into (S)-phenylethanol with phosphine-phosphinoxydes
(la-Ld) and diamines (Le-Lf)

| | | | Ruthenium | | | Time | | |
|---|---|---|---|---|---|---|---|---|
| N^{o} | PPO | NN | precursor | Com/Base | Base | [min] | Conv. % | ee % |
| 1 | Lb | Lf | [RuCl₂(COD)]ₙ | 1000/10000 | KO^{t}Bu | 30 | 100 | 43 |
| 2 | Lb | Lf | [RuCl₂(COD)]ₙ | 1000/2000 | KO^{t}Bu | 30 | 98 | 44 |
| 3¹⁾ | — | Lf | [Ru(p-Cym)(Lb)Cl₂] | 1000/2000 | KO^{t}Bu | 60 | 100 | 42 |
| 4 | Lb | Lh | [RuCl₂(COD)]ₙ | 1000/10000 | KO^{t}Bu | 30 | 26 | 2 |
| 5 | La | Lf | [Ru(COD)(C₄H₇)₂] | 1000/0 | — | 60 | 98 | 17 |
| 6* | La | Lh | [Ru(COD)(C₄H₇)₂] | 1000/0 | — | 60 | 1 | — |
| 7 | Lb | Lf | [Ru(COD)(C₄H₇)₂] | 1000/0 | — | 60 | 100 | 45 |
| 8 | Lc | Lf | [Ru(COD)(C₄H₇)₂] | 1000/0 | — | 60 | 96 | 69 |
| 9 | Lc | Lg | [Ru(COD)(C₄H₇)₂] | 1000/0 | — | 60 | 83 | 25 |
| 10 | Ld | Lf | [Ru(COD)(C₄H₇)₂] | 1000/0 | — | 60 | 99 | 34 |
| 11 | Ld | Lg | [Ru(COD)(C₄H₇)₂] | 1000/0 | — | 60 | 99 | 43 |
| 12* | Le | Lf | [Ru(COD)(C₄H₇)₂] | 1000/0 | — | 60 | 19 | 18 |
| 13* | — | Lf | [Ru(COD)(C₄H₇)₂] | 1000/0 | — | 60 | 21 | 19 |

Com/Base: molar ratio in ppm relative to the substrate.
Conv. = conversion (in %, analysed by GC) of acetophenone into phenylethanol after the indicated time. Reaction conditions: H₂ gas (50 bar), 60° C., iPrOH (2M).
*out or the invention's scope.
¹⁾The ligand (P-PO) is present in the precursor.

### Example 2

Catalytic hydrogenation of acetophenone using various invention ruthenium complexes: formation in situ, from [RuCl₂(COD)ₙ with 1,3-bis(diphenylphosphino)propane monoxide (Li) as (P—PO) and diamine ligands (Lj-Ln) as (N—N), the hydrogenation process carried out in the presence of a base.

A typical experimental procedure is as follows:

In a glove box under argon, a Keim autoclave equipped with a magnetic stirring bar was charged with [RuCl₂(COD)]ₙ (0.1 mol %), 1,3-bis(diphenylphosphino)propane monoxide (0.11 mol %), the appropriate diamine (0.11 mol %), KO^{t}Bu (1 mol %) and iPrOH (6 ml). The suspension was stirred for 30 min, then a solution of acetophenone (20 mmol) and n-tridecane (1 mmol) in iPrOH (2 ml) was added, followed by more iPrOH (2×1 ml). The autoclave was then pressurized with hydrogen gas at 50 bar and placed in a thermostated oil bath set at 60° C. After one hour, the autoclave was removed from the oil bath, and cooled in a cold-water bath. The autoclave was vented and opened, an aliquot (0.2 ml) was taken, diluted with MTBE (5 ml), washed with aq. sat. NH₄Cl (5 ml) and filtered over a plug of celite® 560 and analyzed by GC (DB-Wax).

TABLE 4
Hydrogenation of acetophenone into phenylethanol with 1,3-bis(diphenyl-
phosphino)propane monoxide (Li) and diamines (Lj-Ln.)

| | | | | | ROH | ee |
|---|---|---|---|---|---|---|
| N^{o} | P—PO | N—N | [Ru] | Com/Base | [%] | [%] |
| 1 | 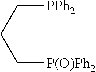 Li | 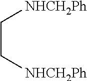 Lj | [RuCl₂(COD)]ₙ | 1000/10000 | >99 | — |
| 2 | 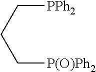 Li | 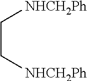 Lj | [Ru(O₂CCF₃)₂(COD)] | 1000/10000 | 84 | — |
| 3 | 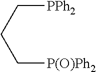 Li | 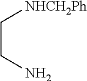 Lk | [RuCl₂(COD)]ₙ | 1000/10000 | 98 | — |
| 4 | 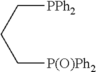 Li | 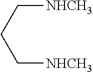 Ll | [RuCl₂(COD)]ₙ | 1000/10000 | 70 | — |
| 5 | 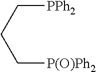 Li | 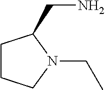 Lm | [RuCl₂(COD)]ₙ | 1000/10000 | 66 | 12 (R) |
| 6 | 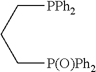 Li | 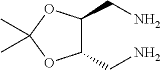 Ln | [RuCl₂(COD)]ₙ | 1000/10000 | 91 | 10 (R) |

Com/Base: molar ratio in ppm relative to the substrate.
ROH = amount of phenylethanol in % analysed by GC after 1 h.
Reaction conditions: H₂ gas (50 bar), 60° C., iPrOH (2M), 1 h.
Ph = phenyl.

### Example 3

Catalytic hydrogenation of acetophenone using various invention ruthenium complexes: formation in situ, from [Ru(C₄H₇)₂(COD)] with phosphine-phosphinoxide ligands as (P—PO) and diamine ligands as (N—N), the hydrogenation process carried out in the absence of a base.

A typical experimental procedure is as follows:

In a glove box under argon, a heavy wall screw cap tube equipped with a magnetic stirring bar was charged with the ruthenium precursor (0.1 mol %), the appropriate bis-diphenylphosphine monooxide (0.11 mol %), the appropriate diamine (0.11 mol %), and iPrOH (3 ml). The suspension was stirred for 60 min in an oil bath at 60° C., then the solution was transferred in a Keim autoclave equipped with a magnetic stirring bar, then a solution of acetophenone (20 mmol) and n-tridecane (1 mmol) in iPrOH (2 ml) was added, followed by more iPrOH (5×1 ml). The autoclave was then pressurized with hydrogen gas at 50 bar and placed in a thermostated oil bath set at 60° C. After one hour, the autoclave was removed from the oil bath, and cooled in a cold-water bath. The autoclave was vented and opened, an aliquot (0.2 ml) was taken, diluted with MTBE (5 ml), filtered over a plug of celite® 560 and analyzed by GC (DB-Wax).

TABLE 5
Hydrogenation of acetophenone into polyethanol with phosphine-
phosphinoxide ligands as (P—PO) and diamine ligands as (N—N) in the absence
of a base

| | | | | ROH | ee |
|---|---|---|---|---|---|
| N^{o} | P—PO | N—N | [Ru] | [%] | [%] |
| 1 | 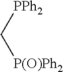 La | 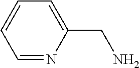 Lp | [Ru(C₄H₇)₂(COD)] | 70 | — |
| 2 | 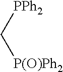 La | 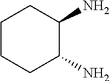 (rac)-Lq | [Ru(C₄H₇)₂(COD)] | 98 | — |
| 3 | 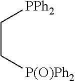 Lb | 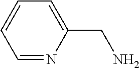 Lp | [Ru(C₄H₇)₂(COD)] | 95 | — |
| 4 | 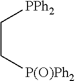 Lb | 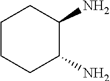 (rac)-Lq | [Ru(C₄H₇)₂(COD)] | >99 | — |
| 5 | 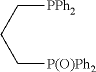 Li | 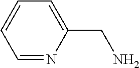 Lp | [Ru(C₄H₇)₂(COD)] | >99 | — |
| 6 | 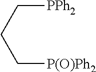 Li | 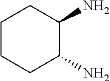 (S,S)-Lq | [Ru(C₄H₇)₂(COD)] | >99 | 21 (R) |
| 7 | 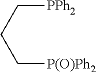 Li | 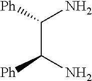 Lg | [Ru(C₄H₇)₂(COD)] | >99 | 33 (R) |
| 8 | 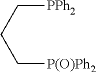 Li | 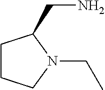 Lm | [Ru(C₄H₇)₂(COD)] | 18 | 45 (R) |
| 9 | 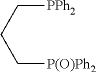 Li | 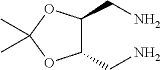 Ln | [Ru(C₄H₇)₂(COD)] | 13 | 8 (R) |
| 10 | 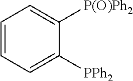 Lo | 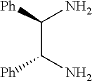 Lf | [Ru(C₄H₇)₂(COD)] | 37 | 30 (S) |
| 11 | 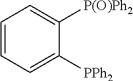 Lo | 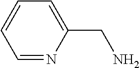 Lp | [Ru(C₄H₇)₂(COD)] | 10 | — |

ROH = amount of phenylethanol in % analysed by GC after 1 h.
Reaction conditions: ruthenium complex (1000 ppm relative to the substrate), H₂ gas (50 bar), 60° C., iPrOH (2M), 1 h.
Ph = phenyl.

### Example 4

Catalytic hydrogenation of various ketone and aldehyde acetophenone using various invention ruthenium complexes: formation in situ, from [Ru(C₄H₇)₂(COD)] with phosphine-phosphinoxide ligands as (P—PO) and diamine ligands as (N—N), the hydrogenation process carried out in the absence of a base.

A typical experimental procedure is as follows:

In a glove box under argon, a heavy wall screw cap tube equipped with a magnetic stirring bar was charged with [Ru(C₄H₇)₂(COD)] (0.1 mol %), the appropriate bis-diphenylphosphine monooxide (0.11 mol %), the appropriate diamine (0.11 mol %), and iPrOH (3 ml). The suspension was stirred for 60 min in an oil bath at 60° C., then the solution was transferred in a Keim autoclave equipped with a magnetic stirring bar, then a solution of the appropriate substrate (20 mmol) in iPrOH (2 ml) was added, followed by more iPrOH (5×1 ml). The autoclave was then pressurized with hydrogen gas at 50 bar and placed in a thermostated oil bath set at 60° C. After one hour, the autoclave was removed from the oil bath, and cooled in a cold-water bath. The autoclave was vented and opened; an aliquot (0.1 ml) was taken, diluted with MTBE (3 ml), filtered over a plug of celite® 560 and analyzed by GC (DB-Wax). The reaction mixture was concentrated under vacuum and the desired alcohol isolated by flash chromatography or distillation.

TABLE 6
Hydrogenation of various ketone and aldehyde into their corresponding alcohol
with [Ru(C₄H₇)₂(COD)] and various diphenylphosphine-monooxyde and
various diamines in the absence of a base

| | | | | ROH | Yield |
|---|---|---|---|---|---|
| N^{o} | P—PO | N—N | Substrate | [%] | [%] |
| 1 | 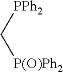 La | 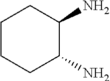 (S,S)-Lq | 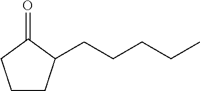 | cis: 14 trans: 1 | — |
| 2 | 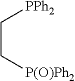 Lb | 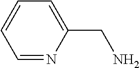 Lp | 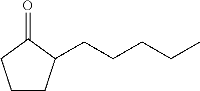 | cis: 35 trans: 5 | — |
| 3 | 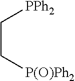 Lb | 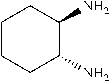 (S,S)-Lq | 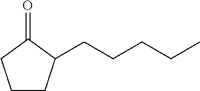 | cis: 85 (15% ee) trans: 5 (5% ee) | — |
| 4 | 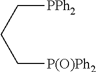 Li | 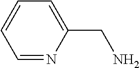 Lp | 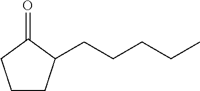 | cis: 95 trans: 4 | — |
| 5 | 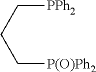 Li | 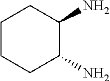 (S,S)-Lq | 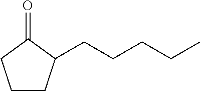 | cis: 93 (8% ee) trans: 6 (2% ee) | — |
| 6¹⁾ | 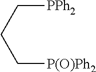 Li | 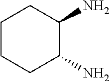 (R,R)-Lq | 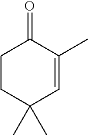 | 96²⁾ | 90 (19% ee) |
| 7³⁾ | 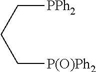 Li | 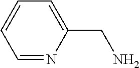 Lp | 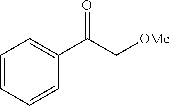 | 16 | — |
| 8 | 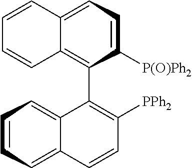 Lc | 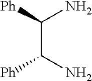 Lf | 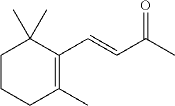 | 46 | 43 (45% ee) |
| 9 | 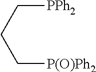 Li | 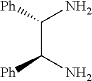 Lg | 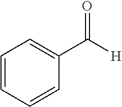 | 20 | — |

ROH = amount of alcohol in % analysed by GC after 1 h. Yield of the isolated desired product after flash chromatography or distillation.
¹⁾The reaction was run for 90 min.
²⁾The saturated alcohol was also formed (GC: 4%).
³⁾The reaction was run for 240 min.
Reaction conditions: ruthenium complex (1000 ppm relative to the substrate), H₂ gas (50 bar), 60° C., iPrOH (2M), 1 h otherwise indicated.
Ph = phenyl.

### Example 5

Catalytic hydrogenation of various ketone and aldehyde acetophenone using various invention ruthenium complexes: formation in situ, from [RuCl₂(COD)] with phosphine-phosphinoxide ligands as (P—PO) and diamine ligands as (N—N), the hydrogenation process carried out in the presence of a base.

A typical experimental procedure is as follows:

In a glove box under argon, a Keim autoclave equipped with a magnetic stirring bar was charged with [RuCl₂(COD)] (0.1 mol %), the appropriate bis-diphenylphosphine monooxide (0.11 mol %), the appropriate diamine (0.11 mol %), KO^{t}Bu (1 mol %) and iPrOH (6 ml). The suspension was stirred for 30 min, then a solution of the appropriate substrate (20 mmol) in iPrOH (2 ml) was added, followed by more iPrOH (2×1 ml). The autoclave was then pressurized with hydrogen gas at 50 bar and placed in a thermostated oil bath set at 60° C. After the indicated time, the autoclave was removed from the oil bath, and cooled in a cold-water bath. The autoclave was vented and opened, an aliquot (0.1 ml) was taken, diluted with MTBE (3 ml), washed with aq. sat. NH₄Cl (3 mL) and filtered over a plug of celite® 560 and analyzed by GC (DB-Wax). The reaction mixture was concentrated under vacuum and the desired alcohol isolated by flash chromatography or distillation.

TABLE 7
Hydrogenation of various ketone, aldehyde and ester into their corresponding
alcohol with [RuCl₂(COD)] and various diphenylphosphine-monooxyde ligands
and various diamine ligand in the presence of a base

| | | | | ROH [%]/t | Yield |
|---|---|---|---|---|---|
| N^{o} | PPO | NN | Substrate | [min] | [%] |
| 1 | 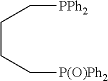 Lr | 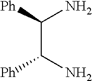 Lf | 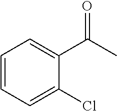 | >99/30 | 88 (39% ee) |
| 2 | 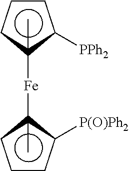 Ls | 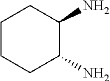 (R,R)-Lq | 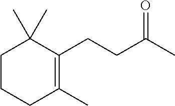 | >99/60 | 96 |
| 3 | 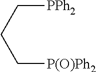 Li | 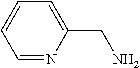 Lp | 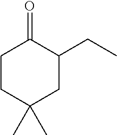 | >99/30 cis/trans: 86/14 | 96 |
| 4 | 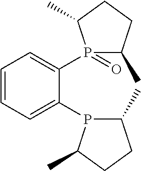 Ld | 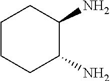 (R,R)-Lq | 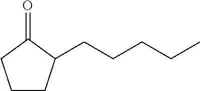 | >99/90 cis/trans: 72/28 | 96 |
| 5 | 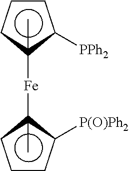 Ls | 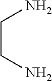 Lt | 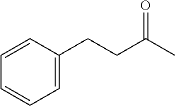 | >99/60 | 92 |
| 6 | 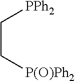 Lb | 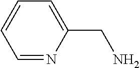 Lp | 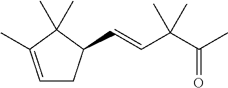 | 97/90 | 94 |
| 7 | 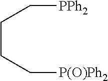 Lr | 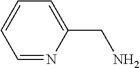 Lp | 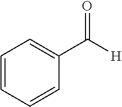 | 37/30 | — |
| 8¹⁾ | 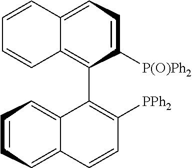 Lc | 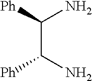 Lf | 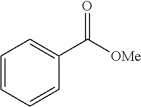 | 17/1200 | — |

ROH = amount of alcohol in % analysed by GC after the indicated time. Yield of isolated desired product after flash chromatography or distillation.
¹⁾[Ru(CF₃COO)₂(COD)], NaOMe in THF were used here: com/base: 500/50000 ppm relative to the substrate.
Reaction conditions: com/base: 1000/10000 ppm relative to the substrate, H₂ gas (50 bar), 60° C., iPrOH (2M).
Ph = phenyl.

1.-12. (canceled)

13. A process for the reduction by hydrogenation of a C₃-C₇₀ substrate containing one or two ketones, aldehydes, ester or lactone functional groups into its corresponding alcohol or diol, which comprises conducting the process in the presence of:
at least one catalyst or pre-catalyst in the form of a ruthenium complex comprising:
a C₂₋₄₀ diamino bidentate ligand (N—N) wherein at least one of the amino groups is a secondary or primary amine with the nitrogen atom of the amine bound to hydrogen atoms or sp³ carbon atoms;
a C₆₋₅₀ phosphine-(phosphine oxide) bidentate ligand (P—PO ligand); and
optionally, a base.

14. The process according to claim 13, wherein the ruthenium complex is of formula
[Ru(P—PO)(N—N)(S)₂₋ᵣYᵣ](Z)₂₋ᵣ (A)
wherein r represents 0, 1 or 2;
S represents a neutral C₁-C₂₆ neutral monodentate ligand;
(P—PO) and (N—N) each represents a ligand;
each Y represents, simultaneously or independently, a hydrogen atom, a hydroxyl, a C₁-C₁₀alkoxyl, a halogen atom, or an C₃-C₁₅ allyl group; and
each Z represents, simultaneously or independently, ClO₄⁻, BF₄—, PF₆—, SbCl₆—, AsCl₆—, SbF₆—, AsF₆—, R^{d}SO₃⁻ wherein R^{d} is a chlorine of fluoride atom or an C₁-C₈ alkyl, aryl, fluoroalkyl or fluoroaryl group, or BR^{e}₄⁻ wherein R^{e} is a phenyl group optionally substituted by one to five halide, methyl or CF₃ groups.

15. The process according to claim 13, wherein the ruthenium complex is of formula
[Ru(P—PO)(N—N)Y₂] (A′).

16. The process according to claim 14, wherein the diamino bidentate ligand (N—N) is a racemic or an optically active compound of formula 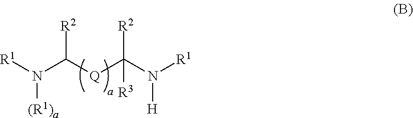
wherein each a, simultaneously or independently, represents 0 or 1;
each R¹, taken separately, represents, simultaneously or independently, a hydrogen atom or a C₁₋₁₀ alkyl or alkenyl group optionally substituted; or two R¹, taken together, may form a saturated heterocycle containing 5 to 10 atoms and including the atoms to which the R¹ are bonded, with the heterocycle being optionally substituted;
R² and R³, taken separately, represent, simultaneously or independently, a hydrogen atom, a C₁₋₁₀ alkyl or alkenyl group optionally substituted or a C₆₋₁₀ aromatic group optionally substituted;
with R¹ and an adjacent R², taken together, optionally forming a saturated or aromatic heterocycle containing 5 to 12 atoms and including the atoms to which the R¹ and R² are bonded, and being optionally substituted and optionally containing one additional nitrogen, oxygen or sulfur atom; with two R², or R² and R³, taken together, optionally forming a saturated or unsaturated ring having 5 to 12 atoms and including the carbon atom to which the R² or R³ groups are bonded, with the ring being optionally substituted and optionally containing one additional nitrogen, oxygen or sulfur atom; and
Q represents;
a group of formula 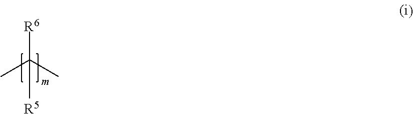
wherein:
m is 1 or 2, and
R⁵ and R⁶ represent, simultaneously or independently, a hydrogen atom, a C₁₋₁₀ alkyl or alkenyl group optionally substituted, a C₆₋₁₀ aromatic group optionally substituted, or an OR⁷ group, R⁷ being a C₁₋₁₀ alkyl or alkenyl group; with two distinct R⁶ or R⁵ groups, or R⁵ or R⁶ and R¹ or R², taken together, optionally forming a C₃₋₁₀ saturated or unsaturated ring that is optionally substituted, including the atoms to which the R⁶, R⁵, R¹ or R² groups are bonded, and optionally containing one or two additional nitrogen, oxygen or sulfur atoms; or
a C₁₀-C₁₆ metallocenediyl, a 2,2′-diphenyl, a 1,1′-binaphthalene-2,2′-diyl, a benzenediyl, a naphthalenediyl, a 4,12-[2:2]-paracyclophanediyl, a 1,6-spiro[4:4]nonanediyl, 3,4-(1-benzyl)-pyrrolidinediyl, 2,3-bicyclo[2:2:1]hept-5-enediyl, 4,6-phenoxazinediyl, 4,5-(9,9-dimethyl)-xanthenediyl, 3,3′-bipyri-4,4′-diyl or 2,2′-(1,1′-bicyclopentyl)-diyl group optionally substituted;
with the optional substituents of R¹, R², R³, R⁵, R⁶ or Q being one, two, three or four groups of i) halogens, ii) C₅₋₁₂ cycloalkyl or cycloalkenyl moieties, iii) C₁₋₁₀ alkoxy, alkyl, alkenyl, polyalkyleneglycols or halo- or perhalo-hydrocarbons, iv) COOR⁴ wherein R⁴ is a C₁₋₆ alkyl, or v) a benzyl group or a fused or non-fused phenyl or indanyl group, with the group being optionally substituted by one, two or three halogens, C₁₋₈ alkyl, alkoxy, amino, nitro, ester, sulfonate or halo- or perhalo-hydrocarbon groups, and with the Q group also being optionally substituted by one or two group of formula O—(CR⁸₂)ₙ—O or O—(CR⁸₂)ₙ—NR⁴ wherein n is 1 or 2 and R⁸ being a hydrogen atom or a C₁₋₄ alkyl group.

17. The process according to claim 16, wherein the diamino bidentate ligand (N—N) is a racemic or an optically active compound of formula 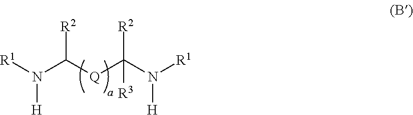
wherein:
a represents 0 or 1; and
each R¹, simultaneously or independently, represents a hydrogen atom or a C₁₋₄ alkyl group optionally substituted;
R² and R³, taken separately, represent, simultaneously or independently, a hydrogen atom, a C₁₋₄ alkyl group optionally substituted or a phenyl group optionally substituted; a R¹ and an adjacent R², taken together, may form a saturated heterocycle containing 5 or 6 atoms and including the atoms to which the R¹ and R² are bonded, and being optionally substituted and optionally containing one additional nitrogen or oxygen atom; two R², or R² and R³, taken together, optionally forming a saturated or unsaturated ring having 5 or 6 atoms and including the atoms to which the R² or R³ groups are bonded, with the ring being optionally substituted and optionally containing one additional oxygen atom; and
Q represents a group of formula 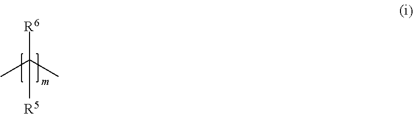
wherein:
m is 1 or 2, and
R⁵ and R⁶ represent, simultaneously or independently, a hydrogen atom, a C₁₋₄ alkyl group optionally substituted or a phenyl group optionally substituted; two distinct R⁶/or R⁵ groups, or R⁶ or R⁵ and a R¹ or R², taken together, may form a C₃₋₆, saturated or unsaturated ring optionally substituted, including the atoms to which the R⁶, R⁵, R¹ or R² groups are bonded and optionally containing one or two additional oxygen atoms; with the optional substituents of R¹, R², R³, R⁵, R⁶ or Q being one or two i) halogen, ii) C₁₋₅ alkyl or alkoxy groups, iii) COOR^{f} wherein R^{f} is a C₁₋₄ alkyl, or v) a benzyl group or a fused or non-fused phenyl group, the group being optionally substituted by one, two or three halogen, C₁₋₄ alkyl or alkoxy groups, esters or sulfonate groups.

18. The process according to claim 16, wherein the diamino bidentate ligand (N—N) is a racemic or an optically active compound of formula 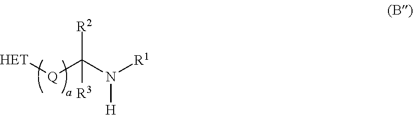
wherein:
a represents 0 or 1; and
R¹ represents a hydrogen atom or a C₁₋₄ alkyl group optionally substituted;
R² and R³, taken separately, represent, simultaneously or independently, a hydrogen atom, a C₁₋₄ alkyl group optionally substituted or a phenyl group optionally substituted; R¹ and R², or R² and R³, taken together, may form a saturated cycle containing 5 or 6 atoms and including the atoms to which the R¹, R² or R³ are bonded, and being optionally substituted and optionally containing one additional nitrogen or oxygen atom; and
HET represents a 2-pyridinyl group optionally substituted by one, two or three C₁₋₄ alkyl groups or by a benzyl group or a fused or non-fused phenyl or indanyl group, the group being optionally substituted by one, two or three halogen, C₁₋₄ alkyl, alkoxy, amino, nitro, ester or sulfonate groups; and
Q represents a group of formula 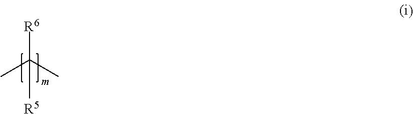
wherein:
m is 1 or 2, and
R⁵ and R⁶ represent, simultaneously or independently, a hydrogen atom, a C₁₋₄ alkyl group optionally substituted or a phenyl group optionally substituted; two distinct R⁶ or R⁵ groups, or R⁶ or R⁵ and R¹ or R² or R⁹ or R^{9′}, taken together, may form a C₃₋₆, saturated or unsaturated ring optionally substituted, including the atoms to which the R⁶, R⁵, R¹ or R² groups are bonded and optionally containing one or two additional oxygen atoms;
with the optional substituents of R¹, R², R³, R⁵, R⁶ or Q being one or two i) halogen, ii) C₁₋₅ alkyl or alkoxy groups, iii) COOR^{f} wherein R^{f} is a C₁₋₄ alkyl, or iv) a benzyl group or a fused or non-fused phenyl group, the group being optionally substituted by one, two or three halogen, C₁-₄ alkyl or alkoxy groups, esters or sulfonate groups.

19. The process according to claim 14, wherein the bidentate ligand (P—PO) is a racemic or an optically active compound of formula 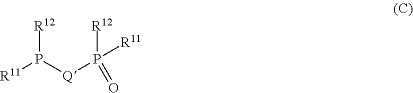
wherein R¹¹ and R¹², when taken separately, represent, simultaneously or independently, a C₁₋C₈ alkyl or alkenyl group optionally substituted or a C₆₋C₁₀ aromatic group optionally substituted; or the R¹¹ and R¹² bounded to the same phosphorous atom, when taken together, may form a saturated or unsaturated ring optionally substituted, having 4 to 8 atoms and including the phosphorus atom to which the R¹¹ and R¹² groups are bonded; and
Q′ represents
a group of formula 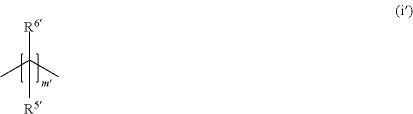
wherein:
m′ is 1, 2, 3 or 4 and
R^{5′} and R^{6′} represent, simultaneously or independently, a hydrogen atom, a C₁₋C₁₀ alkyl or alkenyl group optionally substituted or a C₆₋₁₀ aromatic group optionally substituted, or an OR^{7′} group, R^{7′} being a linear, branched or cyclic C₁₋₁₀ alkyl or alkenyl group; with two distinct R^{6′} or R^{5′} groups, taken together, optionally forming a C₃ to C₁₀ saturated or unsaturated ring optionally substituted, including the atoms to which the R^{6′} or R^{5′} groups are bonded, and optionally containing one or two additional nitrogen or oxygen atoms; or
a C₁₀-C₁₆ metallocenediyl, a 2,2′-diphenyl, a 1,1′-binaphthalene-2,2′-diyl, a benzenediyl, a naphthalenediyl, a 4,12-[2:2]-paracyclophanediyl, a 1,6-spiro[4:4]nonanediyl, 3,4-(1-benzyl)-pyrrolidinediyl, 2,3-bicyclo[2:2:1]hept-5-enediyl, 4,6-phenoxazinediyl, 4,5-(9,9-dimethyl)-xanthenediyl, 3,3′-bipyri-4,4′-diyl or 2,2′-(1,1′-bicyclopentyl)-diyl group optionally substituted;
with the optional substituents of R^{5′}, R^{6′}, R¹¹ and R¹² being one to five halogens, or one, two or three i) C₁₋₁₀ alkyl alkenyl, alkoxy, polyalkyleneglycols groups or halo- or perhalo-hydrocarbon, amine or quaternary amine groups, ii) COOR^{h} wherein R^{h} is a C₁₋₆ alkyl group, iii) C₅₋₁₂ cycloalkyl or cycloalkenyl group, iv) NO₂ group, or v) a benzyl group or a fused or non-fused phenyl, indanyl or naphthyl group, the group being optionally substituted by one, two or three halogen, C₁₋₈ alkyl, alkoxy, amino, nitro, ester, sulfonate or halo- or perhalo-hydrocarbon groups, and the Q′ group may be also be substituted by one or two groups of formula O—(CR^{8′}₂)_{n′}—O or O—(CR^{8′}₂)_{n′}—NR^{4′} wherein n′ is 1 or 2, R^{4′} being a C₁₋₄ alkyl group and R^{8′} being a hydrogen atom or a C₁₋₄ alkyl group.

20. The process according to claim 19, wherein the bidentate ligand (P—PO) is a racemic or an optically active compound of formula (C) wherein
R¹¹ and R¹² represent, simultaneously or independently, a phenyl group optionally substituted; or
the groups R¹¹ and R¹² bounded to the same phosphorous atom, taken together, form a saturated ring optionally substituted, having 4 to 7 atoms and including the phosphorus atom to which the R¹¹ and R¹² groups are bonded; and
Q′ represents a C₂-C₃ alkanediyl radical optionally substituted, a C₁₀-C₁₂ ferrocenediyl, a 2,2′-diphenyl, a 1,1′-(bis(naphthyl)-2,2′-diyl, a 1,2-benzenediyl or a naphthalenediyl group optionally substituted.

21. The process according to claim 13, wherein the substrate is a C₃₋₃₀ compound of formula 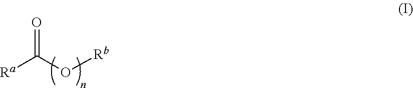
wherein:
n represents 0 or 1;
R^{a} represents a hydrogen atom or a R^{b} group;
R^{b} represents a C₁-C₃₀ hydrocarbon group, optionally substituted and optionally comprising one, two, three or four heteroatoms selected from the group consisting of oxygen, nitrogen or halogens; or
R^{a} and R^{b}, taken together, represent a C₃-C₂₀ saturated or unsaturated hydrocarbon group, optionally substituted and optionally comprising one, two, three or four heteroatoms selected from the group consisting of oxygen, nitrogen or halogens; and
the optional substituents of R^{a} and R^{b} are one, two or three halogen, COOR^{c}, OR^{c}, NR^{c}₂ or R^{c} groups, in which R^{c} is a hydrogen atom, a halogenated C₁-C₂ group or a C₁ to C₁₀ cyclic, linear or branched alkyl, or alkenyl group.

22. The process according to claim 13, wherein the substrate is of formula 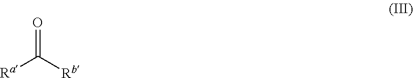
wherein:
R^{a′} represents a hydrogen atom or a C₁₋₄ alkyl or alkenyl group;
R^{b′} represents a C₅-C₁₄ hydrocarbon group, preferably alkyl or alkenyl, optionally substituted and optionally comprising one or two oxygen or nitrogen atoms; or
R^{a′} and R^{b′}, taken together, represent a C₄-C₁₆, hydrocarbon group, preferably alkyl, alkenyl or alkadienyl, optionally substituted and optionally substituted and optionally comprising one or two oxygen or nitrogen atoms; and
the optional substituents of R^{a′} and R^{b′}are one or two OR^{c}, COOR^{c}, CONR^{C}₂ or R^{c} groups, in which R^{c} is a hydrogen atom or a C₁ to C₄ linear or branched alkyl or alkenyl group.

23. The process according to claim 13, wherein the substrate is a:
C₁₁-C₁₈ ketone comprising a trimethyl-cyclohexyl or trimethyl-cyclohexenyl moiety;
C₉-C₁₆ ketone comprising a 2,2,3-trimethyl-cyclopentenyl or 2,2,3-trimethyl-cyclopentyl moiety;
C₁₀-C₁₆ ketone comprising a naphthalenone moiety;
C₅-C₁₄ ketone comprising a cyclopentanone or cyclohexanone moiety; or
C₉-C₁₈ ketone comprising a phenyl moiety.

24. A ruthenium complex of formula
[Ru(P—PO)(N—N)(S)₂₋ᵣYᵣ](Z)₂₋ᵣ (A)
wherein:
r represents 0, 1 or 2;
S represents a neutral C₁-C₂₆ neutral monodentate ligand;
(P—PO) and (N—N) each represents a ligand; and
each Y represents, simultaneously or independently, a hydrogen atom, a hydroxyl, a C₁-C₁₀ alkoxyl, a halogen atom, or an C₃-C₁₅ allyl group; and
each Z represents, simultaneously or independently, ClO₄⁻, BF₄—, PF₆—, SbCl₆—, AsCl₆—, SbF₆—, AsF₆—, a R^{d}SO₃⁻ wherein R^{d} is a chlorine of fluoride atom or an C₁-C₈ alkyl, aryl, fluoroalkyl or fluoroaryl group, or a BR^{e}₄⁻ wherein R^{e} is a phenyl group optionally substituted by one to five groups such as halide atoms or methyl or CF₃ groups;
wherein a C₂₋₄₀ diamino bidentate ligand (N—N) wherein at least one of the amino groups is a secondary or primary amine with the nitrogen atom of the amine bound to hydrogen atoms or sp³ carbon atoms;
wherein (P—PO) is a C₆₋₅₀ phosphine-(phosphine oxide) bidentate ligand; and
wherein (N—N) is a C₂₋₄₀ diamino bidentate ligand with at least one of the amino groups being a secondary or primary amine with the nitrogen atom of the amine bound to hydrogen atoms or sp³ carbon atoms.

25. The ruthenium complex of claim 24 wherein (P—PO) is a racemic or an optically active compound of formula 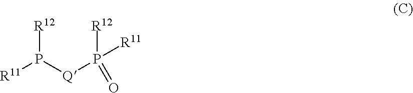
wherein R¹¹ and R¹², when taken separately, represent, simultaneously or independently, a C₁₋₈ alkyl or alkenyl group optionally substituted or a C₆₋₁₀ aromatic group optionally substituted;
or the R¹¹ and R¹² bounded to the same phosphorous atom, when taken together, may form a saturated or unsaturated ring optionally substituted, having 4 to 8 atoms and including the phosphorus atom to which the R¹¹ and R¹² groups are bonded; and
Q′ represents
a group of formula 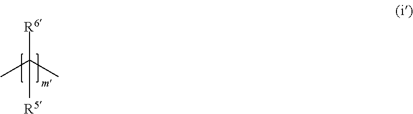
wherein:
m′ is 1, 2, 3 or 4 and
R^{5′} and R^{6′} represent, simultaneously or independently, a hydrogen atom, a C₁₋₁₀ alkyl or alkenyl group optionally substituted or a C₆₋₁₀ aromatic group optionally substituted, or an OR^{7′} group, R^{7′} being a linear, branched or cyclic C₁₋₁₀ alkyl or alkenyl group; with two distinct R^{6′} or R^{5′} groups, taken together, optionally forming a C₃ to C₁₀ saturated or unsaturated ring optionally substituted, including the atoms to which the R^{6′} or R^{5′} groups are bonded, and optionally containing one or two additional nitrogen or oxygen atoms; or
a C₁₀-C₁₆ metallocenediyl, a 2,2′-diphenyl, a 1,1′-binaphthalene-2,2′-diyl, a benzenediyl, a naphthalenediyl, a 4,12-[2:2]-paracyclophanediyl, a 1,6-spiro[4:4]nonanediyl, 3,4-(1-benzyl)-pyrrolidinediyl, 2,3-bicyclo[2:2:1]hept-5-enediyl, 4,6-phenoxazinediyl, 4,5-(9,9-dimethyl)-xanthenediyl, 3,3′-bipyri-4,4′-diyl or 2,2′-(1,1′-bicyclopentyl)-diyl group optionally substituted;
with the optional substituents of R^{5′}, R^{6′}, R¹¹ and R¹² being one to five halogens, or one, two or three i) C₁₋₁₀ alkyl alkenyl, alkoxy, polyalkyleneglycols groups or halo- or perhalo-hydrocarbon, amine or quaternary amine groups, ii) COOR^{h} wherein R^{h} is a C₁₋₆ alkyl group, iii) C₅₋₁₂ cycloalkyl or cycloalkenyl group, iv) NO₂ group, or v) a benzyl group or a fused or non-fused phenyl, indanyl or naphthyl group, the group being optionally substituted by one, two or three halogen, C₁₋₈ alkyl, alkoxy, amino, nitro, ester, sulfonate or halo- or perhalo-hydrocarbon groups, and the Q′ group may be also be substituted by one or two groups of formula O—(CR^{8′}₂)_{n′}—O or O—(CR^{8′}₂)_{n′}—NR^{4′} wherein n′ is 1 or 2, R^{4′} being a C₁₋₄ alkyl group and R^{8′} being a hydrogen atom or a C₁₋₄ alkyl group.

26. The ruthenium complex of claim 25 wherein
(N—N) is a racemic or an optically active compound of formula 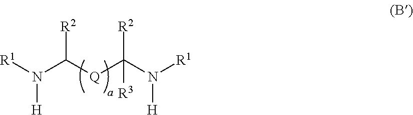
wherein:
a represents 0 or 1; and
each R¹, simultaneously or independently, represents a hydrogen atom or a C₁₋₄ alkyl group optionally substituted;
R² and R³, taken separately, represent, simultaneously or independently, a hydrogen atom, a C₁₋₄ alkyl group optionally substituted or a phenyl group optionally substituted; a R¹ and an adjacent R², taken together, may form a saturated heterocycle containing 5 or 6 atoms and including the atoms to which the R¹ and R² are bonded, and being optionally substituted and optionally containing one additional nitrogen or oxygen atom; two R², or R² and R³, taken together, optionally forming a saturated or unsaturated ring having 5 or 6 atoms and including the atoms to which the R² or R³ groups are bonded, with the ring being optionally substituted and optionally containing one additional oxygen atom; and
Q represents a group of formula 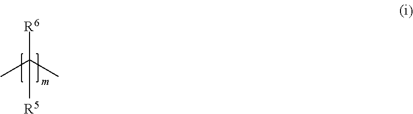
wherein:
m is 1 or 2, and
R⁵ and R⁶ represent, simultaneously or independently, a hydrogen atom, a C₁₋₄ alkyl group optionally substituted or a phenyl group optionally substituted; two distinct R⁶/or R⁵ groups, or R⁶ or R⁵ and a R¹ or R², taken together, may form a C₃₋₆, saturated or unsaturated ring optionally substituted, including the atoms to which the R⁶, R⁵, R¹ or R² groups are bonded and optionally containing one or two additional oxygen atoms; with the optional substituents of R¹, R², R³, R⁵, R⁶ or Q being one or two i) halogen, ii) C₁₋₅ alkyl or alkoxy groups, iii) COOR^{f} wherein R^{f} is a C₁₋₄ alkyl, or v) a benzyl group or a fused or non-fused phenyl group, the group being optionally substituted by one, two or three halogen, C₁₋₄ alkyl or alkoxy groups, esters or sulfonate groups.
